(19) United States
(12) Patent Application Publication
(10) Pub. No.: US 2020/0129319 A1
(43) Pub. Date: Apr. 30, 2020

(54) KNEE JOINT BRACE AND KNEE JOINT SUPPORTING METHOD USING THE SAME

(71) Applicants: BEIJING BOE OPTOELECTRONICS TECHNOLOGY CO.,LTD., Beijing (CN); BOE TECHNOLOGY GROUP CO., LTD., Beijing (CN)

(72) Inventor: Hongyang YU, Beijing (CN)

(21) Appl. No.: 16/452,988

(22) Filed: Jun. 26, 2019

(30) Foreign Application Priority Data
Oct. 24, 2018 (CN) 201821727563.0

(51) Int. Cl. A61F 5/01

(52) U.S. Cl. CPC A61F 5/0123; A61F 2005/0188; A61F 2005/0155; A61F 2005/0137

(57) ABSTRACT

Embodiments of the present disclosure provide a knee joint brace and a knee joint supporting method using the same. The knee joint brace includes: a support frame including two support stands, the two support stands being arranged in parallel with and spaced from each other; at least one adjusting band connected to the support frame; a rotating dual-disc disposed on each of the two support stands, each of the two support stands being capable of bending about the rotating dual-disc; and a supporting band disposed at one end of the support frame and configured to support the support frame in an extending direction of the support stand.

The application claims priority to the Chinese patent application No. 201821727563.0 filed on Oct. 24, 2018, the entire disclosure of which is incorporated herein by reference as part of the present application.

### TECHNICAL FIELD

At least one embodiment of the present disclosure provides a knee joint brace and a knee joint supporting method using the same.

### BACKGROUND

Knee joint brace is a medical instrument for auxiliary fixing, which is wearable by a patient with knee joint injury after a surgical operation and plays an important role in a postoperative recovery of the patient. Existing knee joint braces in the market have substantially the same structure in which a frame is bound by a strapping tape provided with a hook & loop fastener (magic tape).

### SUMMARY

On the first aspect, at least one embodiment of the present disclosure provides a knee joint brace, including:

a support frame including two support stands, the two support stands being arranged in parallel with and spaced from each other;

at least one adjusting band connected to the support frame;

a rotating dual-disc disposed on each of the two support stands, each of the two support stands being capable of bending about the rotating dual-disc; and

a supporting band disposed at one end of the support frame and configured to support the support frame in an extending direction of the support stand.

Optionally, each of the two support stands includes a first keel structure and a second keel structure, the second keel structure is provided with an elastic latch, and the first keel structure is provided with a through hole matched with the latch, so that the first keel structure can be sleeved onto at least a portion of the second keel structure to form the support stand.

Optionally, the second keel structure is provided with a plurality of elastic latches arranged along an extending direction of the second keel structure, and the first keel structure is provided with a plurality of through holes arranged along an extending direction of the first keel structure, and the second keel structure further includes an adjusting block connected to the plurality of elastic latches, the adjusting block is configured to control each of the elastic latches to be projected from or retracted into the second keel structure, so as to be inserted into or separated from one of the through holes to lock the second keel structure with the first keel structure or to separate the second keel structure from the first keel structure.

Optionally, the rotating dual-disc includes a first rotating disc and a second rotating disc, the first rotating disc is disposed on the first keel structure and configured to adjust a bending angle of the first keel structure, and the second rotating disc is disposed on the second keel structure and configured to adjust a bending angle of the second keel structure.

Optionally, the adjusting band is provided with an adjusting device, the adjusting device includes a handle, a rotating shaft connected with the handle, and a magnet spaced apart from the rotating shaft, the rotating shaft is fixed with a buckle structure and a magnetic sheet at opposite sides of the rotating shaft, a surface of the adjusting band facing the buckle structure is provided with a buckle receiver, a rotation of the rotating shaft in a first direction causes an engagement between the buckle structure and the buckle receiver, while a rotation of the rotating shaft in a second direction opposite to the first direction causes a separation of the buckle structure from the buckle receiver, and a magnetic force of the magnet causes the rotation of the rotating shaft in the first direction.

Optionally, the buckle structure is a metal piece, and the buckle receiver is a step-shaped recess.

Optionally, the surface of the adjusting band facing the buckle structure is provided with a plurality of step-shaped recesses.

Optionally, the adjusting device includes a U-shaped case having an upper face and a lower face opposite to each other, and a side face connected with the upper face and the lower face, the side face is opposite to the adjusting band and is provided with the magnet, and the rotating shaft penetrates the upper face and the lower face.

Optionally, the adjusting band is an annular band connected between the two support stands, and a plane of the annular band is intersected with a plane of the support frame.

Optionally, the plane of the annular band is substantially perpendicular to the plane of the support frame.

Optionally, at least two adjusting bands are arranged at an interval along an extending direction of the support frame.

Optionally, the number of the adjusting band connected with the first keel structure is greater than the number of the adjusting band connected with the second keel structure.

Optionally, one surface of the adjusting band is provided with a pressure detecting device, the pressure detecting device is configured to detect pressure data and transmit the pressure data to a designated terminal.

Optionally, the pressure detecting device includes a pressure sensor, a signal line connected with the pressure sensor, a wireless transmitter connected with the signal line, and a battery disposed on the wireless transmitter.

On the second aspect, at least one embodiment of the present disclosure provides a knee joint supporting method by using the knee joint brace of the first aspect, including:

placing the supporting band onto a user's instep; and

fixing the support frame at the user's leg in such a manner that the adjusting band is sleeved onto the leg, the two support stands are attached onto two sides of the leg, the first keel structure corresponds to a thigh and the second keel structure corresponds to a crus.

Optionally, the knee joint supporting method includes: adjusting a bending angle of the first keel structure by the first rotating disc so as to be adapted to a bending of the user's knee joint; and adjusting a bending angle of the second keel structure by the second rotating disc so as to be adapted to a bending of the user's foot with respect to the user's crus.

Optionally, the knee joint supporting method includes: controlling the elastic latch to be projected from or retracted into the second keel structure to be inserted into or separated from the through hole at different positions of the first keel structure by the adjusting block, so as to adjust a length of the support stand and hence a height of the support frame.

Optionally, the knee joint supporting method includes: placing the surface of the adjusting band provided with the pressure detecting device closer to the user's leg; and detecting pressure data of the user's leg by the pressure detecting device and transmitting the pressure data to a designated terminal.

### BRIEF DESCRIPTION OF THE DRAWINGS

In order to clearly illustrate the technical solutions of the embodiments of the present disclosure, the drawings of the embodiments will be briefly described in the following. Apparently, the described embodiments are just a part but not all of the embodiments of the present disclosure. Based on the described embodiments herein, those skilled in the art can obtain other embodiment(s), without any inventive work, which should be within the scope of the present disclosure.

FIG. 1 is a schematic structural diagram of a knee joint brace provided by an embodiment of the present disclosure;

FIG. 2 is a structural side view of a knee joint brace provided by an embodiment of the present disclosure;

FIG. 3A and FIG. 3B are schematic structural diagrams of an adjusting band in a knee joint brace provided by an embodiment of the present disclosure;

FIG. 4 is a partial structural diagram of a knee joint brace provided by an embodiment of the present disclosure; and

FIG. 5 is a schematic structural diagram of a pressure detecting device in a knee joint brace provided by an embodiment of the present disclosure.

### DETAILED DESCRIPTION

In order to make objects, technical details and advantages of the embodiments of the disclosure apparent, the technical solutions of the embodiments will be described in a clearly and fully understandable way in connection with the drawings related to the embodiments of the disclosure. Apparently, the described embodiments are just a part but not all of the embodiments of the disclosure. Based on the described embodiments herein, those skilled in the art can obtain other embodiment(s), without any inventive work, which should be within the scope of the disclosure.

Unless otherwise defined, all the technical and scientific terms used herein have the same meanings as commonly understood by one of ordinary skill in the art to which the present disclosure belongs. The terms “first,” “second,” etc., which are used in the present disclosure, are not intended to indicate any sequence, amount or importance, but distinguish various components. The terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly. “On,” “under,” “right,” “left” and the like are only used to indicate relative position relationship, and when the position of the object which is described is changed, the relative position relationship may be changed accordingly.

The inventor(s) of the present application found out in the research that, the existing knee joint brace mainly involves the following two problems. On one aspect, because the knee joint brace is fixed only by means of a friction force generated from binding and because a human's leg has a shape tapered in a direction pointing to the foot, the knee joint brace is easily to be separated from or fallen off from the human's body for lacking of vertical support. On the other aspect, the hook & loop fastener may be degraded after several times of usage and the fastening effect would be weakened, which may aggravate the above problems.

At least one embodiment of the present disclosure provides a knee joint brace, which can overcome the above-mentioned defects.

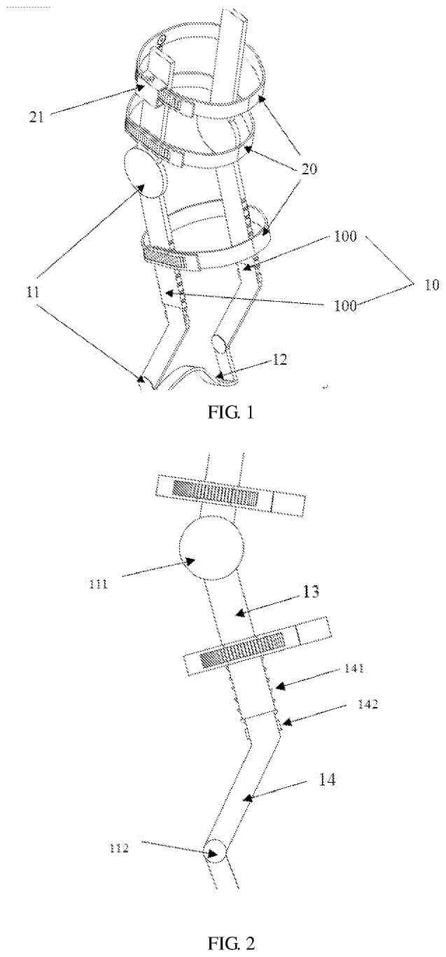

FIG. 1 is a schematic structural diagram of a knee joint brace provided by an embodiment of the present disclosure. Referring to FIG. 1, the knee joint brace includes a support frame 10 and at least one adjusting band 20 connected with the support frame 10. The support frame 10 includes two support stands 100 which are arranged in parallel with each other and are spaced apart from each other. Each of the two support stands 100 is provided with a rotating dual-disc 11 so that the support stand 100 is capable of bending about the rotating dual-disc 11. One end of the support frame 10 is provided with a supporting band 12, and the supporting band 12 can be placed onto a user's instep, so as to support the support frame 10 in an extending direction of the support frame 10 (i.e., the vertical direction).

A human's leg is usually thinner at the crus and thicker at the thigh. As a result, a portion of the knee joint brace corresponding to the crus generally has a dimension smaller than that of a portion corresponding to the thigh, which makes the knee joint brace easy to have a vertical displacement and fall off from the leg. In the embodiment of the present disclosure, one end of the support frame, that is closer to the instep when usage, is provided with a supporting band 12. When worn on a user's leg, the supporting band 12 of the knee joint brace can be placed onto the instep so as to support the support frame 10 in the vertical direction, thereby preventing the knee joint brace from falling off.

In an optional example, a material of the supporting band 12 may be metal or other organic material, without particularly limited in the embodiments of the present disclosure, as long as it allows the supporting band to provide sufficient support effect. In an optional example, the supporting band 12 may have an arc shape to be adaptive to the human's instep, without particularly limited in the embodiments of the present disclosure, as long as it allows the supporting band to provide sufficient support effect and does not result in discomfort of the user.

In an optional example, referring to FIG. 2, the support stand 100 may include a first keel structure 13 and a second keel structure 14; the second keel structure 14 can be at least partly inserted in the first keel structure 13, or the first keel structure 13 can be sleeved onto at least a portion of the second keel structure 14, so as to form the support stand 100. The second keel structure 14 may be provided with an elastic latch 141, while the first keel structure 13 may be provided with a through hole matched with the elastic latch 141 so as to realize fixing/locking the first keel structure with the second keel structure through the engagement between the elastic latch and the through hole.

In an optional example, referring to FIG. 2, the second keel structure 14 is provided with a plurality of elastic latches 141 arranged along an extending direction of the second keel structure 14, and the first keel structure 13 is provided with a plurality of through holes arranged along an extending direction of the first keel structure 13; and the second keel structure 14 further includes an adjusting block 142 connected to the plurality of elastic latches 141, the adjusting block 142 is configured to control each of the elastic latches 141 to be projected from or retracted into the second keel structure 14, so as to be inserted into or separated from one of the through holes to lock the second keel structure 14 with the first keel structure 13 or to separate the second keel structure from the first keel structure 13. In this way, by controlling the elastic latch 141 to be engaged with the through hole at different positions through the adjusting block 142, a length/height of the support stand 100 and hence a length/height of the support frame 10 can be adjusted.

In an optional example, referring to FIG. 2, the rotating dual-disc 11 includes a first rotating disc 111 and a second rotating disc 112. The first rotating disc 111 is disposed on the first keel structure 13, and is configured to adjust a bending angle of the first keel structure 13 when the user's knee joint makes a bending action, so as to be adapted to the bending action of the user's knee joint. The second rotating disc 112 is disposed on the second keel structure 14, and is configured to adjust a bending angle of the second keel structure 14 when the user's foot makes a bending action with respect to the user's crus, so as to be adapted to the bending action of the foot with respect to the crus.

In an optional example, referring to FIG. 1, the adjusting band 20 is an annular band and is connected between the two support stands 100 of the support frame 10; a plane of the adjusting band 20 is intersected with a plane of the support frame 10, for example, the plane of the adjusting band 20 is substantially perpendicular to the plane of the support frame 10; a plurality of adjusting bands 20 may be arranged along the extending direction of the support frame 10 at an interval, for example, at least two adjusting bands 20 may be provided. Although total three adjusting bands 20 are illustrated in FIG. 1, the embodiments of the present disclosure are not limited thereto.

Still referring to FIG. 1, because the first keel structure 13 usually corresponds to the thigh while the second keel structure 14 corresponds to the crus during usage, the number of the adjusting band 20 disposed on the first keel structure 13 may be greater than the number of the adjusting band 20 disposed on the second keel structure 14, without limited the embodiments of the present disclosure thereto.

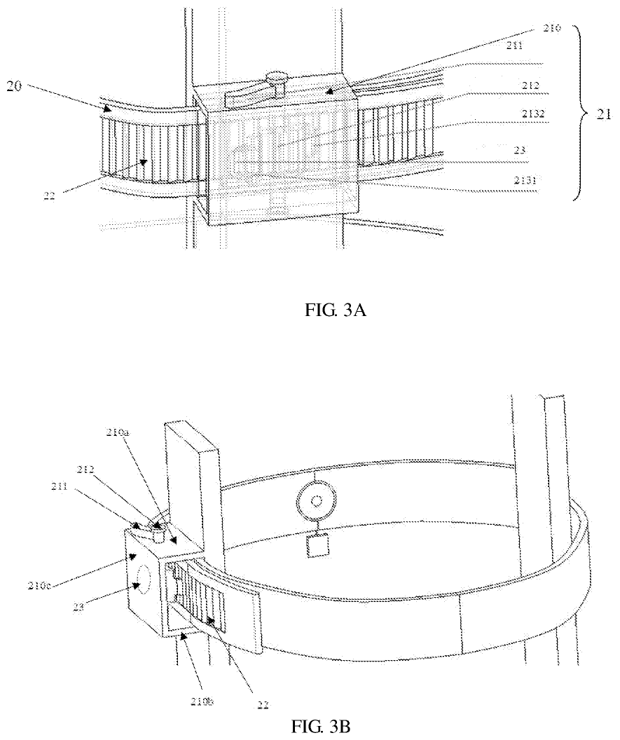

In an optional example, referring to FIG. 3A and FIG. 3B, the adjusting band 20 is provided with an adjusting device 21. The adjusting device 21 includes a handle 211, a rotating shaft 212 connected with the handle 211, and a magnet 23 spaced apart from the rotating shaft 212, for example, a circular magnet 23. The rotating shaft 212 is fixed with a buckle structure 2132 and a magnetic sheet 2131 at opposite sides of the rotating shaft 212; a surface of the adjusting band 20 facing the buckle structure 2132 is provided with a buckle receiver 22. The adjusting device 21 is configured in such a manner that a rotation of the rotating shaft in a first direction causes an engagement between the buckle structure and the buckle receiver, while a rotation of the rotating shaft in a second direction opposite to the first direction causes a separation of the buckle structure from the buckle receiver, and a magnetic force of the magnet causes the rotation of the rotating shaft in the first direction.

In an optional example, referring to FIG. 3A and FIG. 3B, the adjusting device 21 includes a U-shaped case 210 having an upper face 210a and a lower face 210b opposite to each other, and a side face 210c connected with the upper face 210a and the lower face 210b; the side face 210c is opposite to the adjusting band 20 and is provided with the magnet 23; the rotating shaft 212 penetrates the upper face 210a and the lower face 210b. It should be noted that the magnet 23 is illustrated in FIG. 3B using dashed line to indicate that the magnet 23 is located at an inner side of the side surface 210c and is not visible from the outside.

In an optional example, the buckle structure is a metal piece 2132, and the buckle receiver is a step-shaped recess 22. For example, the surface of the adjusting band facing the buckle structure is provided with a plurality of step-shaped recesses to provide different perimeters for the adjusting band.

As above, according to the embodiment of the present disclosure, the adjusting device 21 adopts a combination of mechanical engagement and magnetic engagement in which only the mechanical engagement achieves a locking effect, with the working principle described as follows. When the handle 211 is not applied with an external force, the magnetic sheet 2131 will be attracted by the magnet 23 and rotated in a first direction (e.g., clockwise) to be engaged with the magnet 23, which drives the metal piece 2132 fixed at the opposite side of the rotating shaft 212 to be rotated in the first direction (e.g., clockwise) and engaged with (e.g., snap-fitted into) the recess 22 on the adjusting band 20 so as to mechanically lock the adjusting band 20; in this way, a binding length of the adjusting band 20 (e.g., a diameter of a ring-shaped adjusting band) is fixed, and a binding effect of the knee joint brace is provided. When the handle 211 is applied with an external force to rotate the magnetic sheet 2131 in a second direction (e.g., anticlockwise) opposite to the first direction, the magnetic sheet 2131 will be separated from the magnet 23, which drives the metal piece 2132 fixed at the opposite side of the rotating shaft 212 to be rotated in the second direction (e.g., anticlockwise) and separated from the recess 22 on the adjusting band, so as to unlock the adjusting band 20; at this moment, the binding length (e.g., a diameter of a ring-shaped adjusting band) of the adjusting band may be adjusted by, for example, pulling the adjusting band, so as to adapt to the size of the user's leg, and to obtain expected binding effect.

In an optional example, referring to FIG. 1, only the uppermost adjusting band 20 is provided with the adjusting device 21, while the remaining two are not. However, the embodiments of the present disclosure are not limited thereto. In actual application, the adjusting device may be provided on every adjusting band to provide stable and tight binding effect of the knee joint brace with respect to the leg.

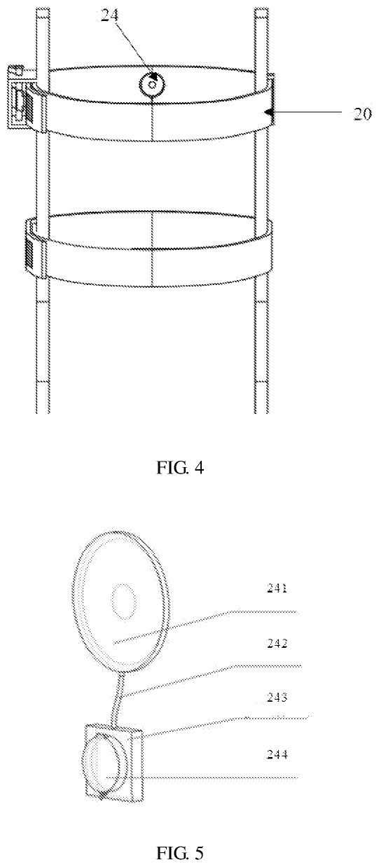

In an optional example, referring to FIG. 4, a surface of the adjusting band 20, that is closer to the leg during usage, is provided with a pressure detecting device 24, the pressure detecting device 24 is configured to detect pressure data of the leg and transmit the pressure data to a designated terminal.

In an optional example, referring to FIG. 5, the pressure detecting device 24 includes a pressure sensor 241, a signal line 242 connected with the pressure sensor 241, a wireless transmitter 243 connected with the signal line 242, and a battery 244 disposed on the wireless transmitter 243. For example, the battery 244 is a button cell.

The knee injury would result in atrophy of thigh muscle, and an exercise of the thigh muscle is very important for the postoperative recovery of the patient. In the embodiments of the present disclosure, the pressure detecting device is provided at an inner side (i.e., the side attached with the leg) of the adjusting band so as to detect pressure data of the leg in real time, and the pressure data is transmitted to the designated terminal in a wireless way, which enables the designated terminal to analyze the pressure data of the patient's leg by utilizing functions of the designated terminal and obtain a recovery condition of the thigh muscle for the user to make exercise plans.

Therefore, the embodiments of the present disclosure provide a knee joint brace including a support frame and at least one adjusting band connected with the support frame; the support frame is provided with a rotating dual-disc so that the support frame is capable of bending about the rotating dual-disc; one end of the support frame is provided with a support band which can be placed onto the user's instep and support the support frame in a vertical direction. For usage, placing the supporting band onto a user's instep, and fixing the support frame at the user's leg in such a manner that the adjusting band is sleeved onto the leg, the two support stands are attached onto two sides of the leg, the first keel structure corresponds to a thigh and the second keel structure corresponds to a crus. At this moment, the supporting band will provide a vertical support for the support frame and prevent the support frame from falling off in the vertical direction. During usage, the length of the adjusting band is adjustable by the adjusting device thereon, to obtain desired binding effect, and the height of the support frame is adjustable by the adjusting block thereon, to obtain desired supporting effect.

The above are only specific implementations of the present disclosure, the protection scope of the present disclosure is not limited thereto. Any modifications or substitutions easily occur to those skilled in the art within the technical scope of the present disclosure should be fallen within the protection scope of the present disclosure. Therefore, the protection scope of the present disclosure should be based on the protection scope of the claims and the equivalents thereof.

What is claimed is:

1. A knee joint brace, comprising:
a support frame comprising two support stands, the two support stands being arranged in parallel with and spaced from each other;
at least one adjusting band connected to the support frame;
a rotating dual-disc disposed on each of the two support stands, each of the two support stands being capable of bending about the rotating dual-disc; and
a supporting band disposed at one end of the support frame and configured to support the support frame in an extending direction of the support stand.

2. The knee joint brace according to claim 1, wherein each of the two support stands comprises a first keel structure and a second keel structure,
the second keel structure is provided with an elastic latch, and the first keel structure is provided with a through hole matched with the latch, so that the first keel structure can be sleeved onto at least a portion of the second keel structure to form the support stand.

3. The knee joint brace according to claim 2, wherein the second keel structure is provided with a plurality of elastic latches arranged along an extending direction of the second keel structure, and the first keel structure is provided with a plurality of through holes arranged along an extending direction of the first keel structure, and
the second keel structure further comprises an adjusting block connected to the plurality of elastic latches, the adjusting block is configured to control each of the elastic latches to be projected from or retracted into the second keel structure, so as to be inserted into or separated from one of the through holes to lock the second keel structure with the first keel structure or to separate the second keel structure from the first keel structure.

4. The knee joint brace according to claim 2, wherein the rotating dual-disc comprises a first rotating disc and a second rotating disc,
the first rotating disc is disposed on the first keel structure and configured to adjust a bending angle of the first keel structure, and
the second rotating disc is disposed on the second keel structure and configured to adjust a bending angle of the second keel structure.

5. The knee joint brace according to claim 1, wherein the adjusting band is provided with an adjusting device,
the adjusting device comprises a handle, a rotating shaft connected with the handle, and a magnet spaced apart from the rotating shaft,
the rotating shaft is fixed with a buckle structure and a magnetic sheet at opposite sides of the rotating shaft,
a surface of the adjusting band facing the buckle structure is provided with a buckle receiver,
a rotation of the rotating shaft in a first direction causes an engagement between the buckle structure and the buckle receiver, while a rotation of the rotating shaft in a second direction opposite to the first direction causes a separation of the buckle structure from the buckle receiver, and
a magnetic force of the magnet causes the rotation of the rotating shaft in the first direction.

6. The knee joint brace according to claim 5, wherein the buckle structure is a metal piece, and the buckle receiver is a step-shaped recess.

7. The knee joint brace according to claim 6, wherein the surface of the adjusting band facing the buckle structure is provided with a plurality of step-shaped recesses.

8. The knee joint brace according to claim 5, wherein the adjusting device comprises a U-shaped case having an upper face and a lower face opposite to each other, and a side face connected with the upper face and the lower face,
the side face is opposite to the adjusting band and is provided with the magnet, and
the rotating shaft penetrates the upper face and the lower face.

9. The knee joint brace according to claim 1, wherein the adjusting band is an annular band connected between the two support stands, and a plane of the annular band is intersected with a plane of the support frame.

10. The knee joint brace according to claim 9, wherein the plane of the annular band is substantially perpendicular to the plane of the support frame.

11. The knee joint brace according to claim 2, wherein at least two adjusting bands are arranged at an interval along an extending direction of the support frame.

12. The knee joint brace according to claim 11, wherein the number of the adjusting band connected with the first keel structure is greater than the number of the adjusting band connected with the second keel structure.

13. The knee joint brace according to claim 1, wherein one surface of the adjusting band is provided with a pressure detecting device, the pressure detecting device is configured to detect pressure data and transmit the pressure data to a designated terminal.

14. The knee joint brace according to claim 13, wherein the pressure detecting device comprises a pressure sensor, a signal line connected with the pressure sensor, a wireless transmitter connected with the signal line, and a battery disposed on the wireless transmitter.

15. A knee joint supporting method by using the knee joint brace according to claim 1, wherein each of the two support stands comprises a first keel structure and a second keel structure,
the knee joint supporting method comprises:
placing the supporting band onto a user's instep; and
fixing the support frame at the user's leg in such a manner that the adjusting band is sleeved onto the leg, the two support stands are attached onto two sides of the leg respectively, the first keel structure corresponds to a thigh and the second keel structure corresponds to a crus.

16. The knee joint supporting method according to claim 15, wherein the rotating dual-disc comprises a first rotating disc and a second rotating disc, the first rotating disc is disposed on the first keel structure, the second rotating disc is disposed on the second keel structure,
the knee joint supporting method further comprises:
adjusting a bending angle of the first keel structure by the first rotating disc so as to be adapted to a bending of the user's knee joint; and
adjusting a bending angle of the second keel structure by the second rotating disc so as to be adapted to a bending of the user's foot with respect to the user's crus.

17. The knee joint supporting method according to claim 15, wherein the second keel structure is provided with a plurality of elastic latches arranged along an extending direction of the second keel structure, the first keel structure is provided with a plurality of through holes arranged along an extending direction of the first keel structure, and the second keel structure further comprises an adjusting block connected to the plurality of elastic latches,
the knee joint supporting method further comprises:
controlling the elastic latch to be projected from or retracted into the second keel structure to be inserted into or separated from the through hole at different positions of the first keel structure by the adjusting block, so as to adjust a length of the support stand and hence a height of the support frame.

18. The knee joint supporting method according to claim 15, wherein one surface of the adjusting band is provided with a pressure detecting device,
the knee joint supporting method further comprises:
placing the surface of the adjusting band provided with the pressure detecting device closer to the user's leg; and
detecting pressure data of the user's leg by the pressure detecting device and transmitting the pressure data to a designated terminal.
